(19) United States
(12) Patent Application Publication
(10) Pub. No.: US 2026/0250258 A1
(43) Pub. Date: Aug. 27, 2026

(54) PROCESS FOR THE SYNTHESIS OF THE COMPOUND 5,7-DIIODO-1-OXASPIRO[2,5]OCTA-4,7- DIEN-6-ONE

(71) Applicants: INNOVERDA, VILLEJUIF (FR); UNIVERSITE PARIS CITE, PARIS (FR); CENTRE NATIONAL DE LA RECHERCHE SCIENTIFIQUE, PARIS CEDEX (FR)

(72) Inventors: Anthony GARCIA, Claye-Souilly (FR); Sylvain DAUNAY, LE PERREUX SUR MARNE (FR); Irene ERDELMEIER, PARIS (FR); Claire FAVE, PARIS (FR); Bernd SCHÖLLHORN, Igny (FR)

(21) Appl. No.: 19/163,402

(22) PCT Filed: Mar. 14, 2024
(86) PCT No.: PCT/EP2024/056787

§ 371 Date: Sep. 9, 2025

(30) Foreign Application Priority Data
Mar. 15, 2023 (FR) FR2302388

(51) Int. Cl. C07D 301/03; C07D 303/32

(52) U.S. Cl. CPC C07D 301/03; C07D 303/32

(57) ABSTRACT

A process for synthesizing the compound 5,7-diiodo-1-oxaspiro[2,5]octa-4,7-dien-6-one. This process for synthesizing the compound 5,7-diiodo-1-oxaspiro[2,5]octa-4,7-dien-6-one includes an oxidation reaction of 4-hydroxy-3,5-diiodobenzyl alcohol with a hypochlorite oxidant, in particular selected from sodium, calcium and potassium hypochlorite. This process enables a significant improvement in yield in a simple and inexpensive way.

### FIELD OF THE INVENTION

The invention concerns a new process for the synthesis of compound 1, 5,7-diiodo-1-oxaspiro[2,5]octa-4,7-dien-6-one, CAS-No. 197230-76-5, which is a potential synthesis intermediate for Levothyrox in particular, as described in Tetrahedron Letters, Vol. 38, No. 40, pp. 6965-6968, 1997.

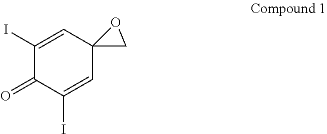 [SMILES: O=C1C(I)=CC2(C=C1I)CO2]

### STATE OF THE ART

Described in Tetrahedron Letters, Vol. 38, No. 40, pp. 6965-6968, 1997 (page 6966, compound 9) is the reaction with the highest yield of compound 1 to date involving the oxidation of 4-Hydroxy-3,5-diiodobenzyl alcohol (compound 2) with sodium bismuthate (conditions shown in Scheme 1)

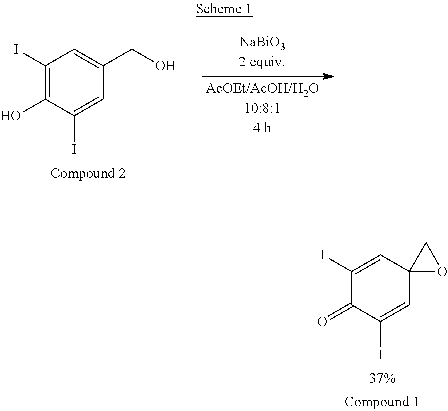 [SMILES: OCc1cc(I)c(O)c(I)c1 ; O=C1C(I)=CC2(C=C1I)CO2]

The desired compound 1 is obtained in 37% yield.

This synthesis route is also described in the patent: Halogenated Phenols for diagnostics, antioxidant protection and drug delivery, WO 2013/010102 A2, with a spiroepoxide yield of 42% (Ex. 3 pages 98 and 99).

The second known synthetic route is described in Organic Letters 2019, 21, 6504-6507, and proceeds in two steps from compound 2:

Step 1: First, a bis-dichloroacetate derivative is formed in 97% yield, see Scheme 2.

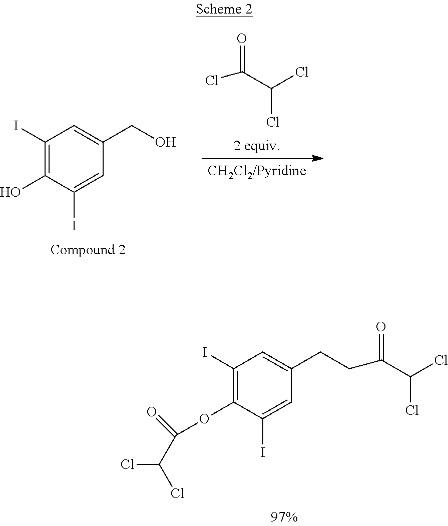 [SMILES: O=C(Cl)C(Cl)Cl ; OCc1cc(I)c(O)c(I)c1 ; O=C(CCc1cc(I)c(OC(=O)C(Cl)Cl)c(I)c1)C(Cl)Cl]

Second step: the intermediate diester is then oxidized with hydrogen peroxide and potassium hydroxide in acetonitrile, giving the desired spiroepoxide 1 in just 20% yield (see Scheme 3, compound 2m on page 6505 of the same article).

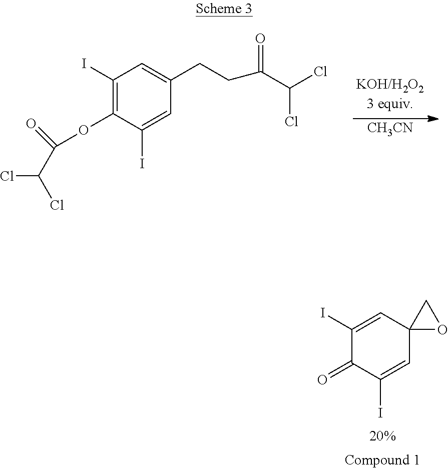 [SMILES: O=C(CCc1cc(I)c(OC(=O)C(Cl)Cl)c(I)c1)C(Cl)Cl ; "CH3CN" ; O=C1C(I)=CC2(C=C1I)CO2]

This process produces the desired compound 1, but the yield is unsatisfactory, and even lower than with sodium bismuthate in direct oxidation. Moreover, it involves toxic solvents (classified as CMR) such as pyridine and dichloromethane.

Interestingly, the non-iodinated analogue (compound 2a in this publication) is obtained by this method in a much higher yield of 66%, indicating that di-iodinated derivatives are more difficult to synthesize in satisfactory yields.

### OBJECT OF THE INVENTION

The aim of the invention is to solve the technical problem of obtaining a direct or single-step synthesis of the compound 5,7-diiodo-1-oxaspiro[2,5]octa-4,7-dien-6-one, in excellent yield, and using a non-toxic reagent(s) and reaction medium.

The aim of the invention is to solve this technical problem with a short reaction time, of the order of a few minutes.

Another aim of the invention is to solve this technical problem using an available, inexpensive substance.

Another aim of the invention is to solve these problems with a simple solution that is safe, reliable, reproducible and can be used on an industrial scale, while avoiding any agents likely to cause environmental problems.

### DETAILED DESCRIPTION OF THE PROCESS ACCORDING TO THE INVENTION

The invention solves these technical problems for the first time using an available starting substance, 4-Hydroxy-3,5-diiodobenzyl alcohol, the synthesis of which from 4-hydroxybenzyl alcohol has also been the subject of a Patent filed by INNOVERDA and published under No. FR 3,113,904B2.

The invention thus concerns a process for the direct one-step synthesis of the compound 5,7-diiodo-1-oxaspiro[2,5]octa-4,7-dien-6-one from 4-hydroxy-3,5-diiodobenzyl alcohol, characterized in that an oxidation reaction of 4-hydroxy-3,5-diiodobenzyl alcohol is carried out with an oxidizing agent comprising or consisting of a hypochlorite, in particular chosen from sodium, calcium, potassium hypochlorite, or mixtures thereof.

According to a particular embodiment of the invention, this oxidation reaction is carried out in a two-phase reaction medium comprising an ether-type polar organic solvent and an aqueous solution.

According to a particular embodiment, the ether-type polar organic solvent is selected from methyl tert-butyl ether (MTBE), tert-amyl methyl ether (TAME), diethyl ether, and methyl cyclopentyl ether, or mixtures thereof.

According to another particular embodiment, the aqueous solution is chosen from water, in particular demineralized water, or an aqueous buffer, in particular an aqueous buffer with a pH between 5 and 8, preferably between 6 and 7. A suitable aqueous buffer is an acetate buffer.

According to yet another particular embodiment, the relative molar ratio of hypochlorite oxidant to 4-hydroxy-3,5-diiodobenzyl alcohol is between 2 and 3, in particular between 2 and 2.5.

According to another particular embodiment, the ratio by volume of the aqueous solution relative to the total volume of the aqueous solution and the ether-type polar organic solvent is between 2 and 21%, in particular between 5 and 12%, still better about 6%.

According to a particular embodiment, the temperature of the reaction medium is between about 0 and room temperature or below room temperature, in particular between about 0 and about 21° C.

According to yet another particular embodiment, the duration of the reaction is between 1 and 15 minutes, in particular between 4 and 10 minutes.

According to yet another particular embodiment, the 4-hydroxy-3,5-diiodobenzyl alcohol is dissolved in the polar organic solvent and the hypochlorite oxidant is dissolved in the aqueous solution.

According to yet another particular embodiment, the molar concentration of the hypochlorite oxidant in the aqueous solution is from 0.5M to 1.5M.

According to yet another particular embodiment, the aqueous solution is added gradually to the polar organic solution, in particular drop by drop, over a period of time of 1 to 15 minutes, in particular 4 to 10 minutes, with stirring, in particular vigorous stirring. Vigorous agitation is understood to mean vortex-creating agitation, as is well known to those skilled in the art. This vortex is generally achieved at a rotational speed of between 800-1200 rpm.

According to yet another particular embodiment of the invention, 4-Hydroxy-3,5-diiodobenzyl alcohol is used directly as the starting material, which is oxidized with sodium or calcium hypochlorite.

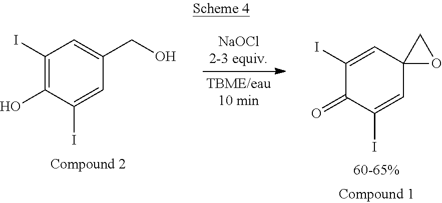 [SMILES: OCc1cc(I)c(O)c(I)c1 ; O=C1C(I)=CC2(C=C1I)CO2]

It is recalled here that compound 2 can be prepared from 4-hydroxybenzylalcohol by the process described in INNOVERDA patent FR-3,113,904B2. With this oxidant, yields of up to 65% can be achieved in surprisingly unexpectedly short reaction times of just a few minutes.

Compared with the known state of the art, the invention brings the following substantial improvements:
1. The yield is increased, practically doubling that of the best method;
2. The process uses an inexpensive oxidant;
3. The reaction time is very short, just a few minutes;
4. The process can be carried out at room temperature.
5. The process does not use toxic heavy metals or rare metals, and the resulting by-products of the oxidizer are non-toxic (H₂O and NaCl).

Thus, the invention implements the use of hypochlorite salts for the synthesis of the compound 1, 5,7-diiodo-1-oxaspiro[2,5]octa-4,7-dien-6-one, a di-iodinated spiro-epoxide, surprisingly and unexpectedly achieving high yields in a few minutes.

The use of sodium and calcium hypochlorite as an oxidant in a chemical reaction is not new.

In 2015, Ishihara et al. describe in Chemistry Letters 2015, 44, 381-383 the use of this oxidant for the oxidative de-aromatization of phenols. Various spiro derivatives are described, such as spirolactones, spirolactams etc.

No spiro-epoxide compounds are described in this publication, nor any examples of reactions with diiodinated phenols.

For example, obtaining the compounds listed in Scheme 5 by oxidation with sodium hypochlorite is described in this publication Chemistry Letters 2015, see page 382:

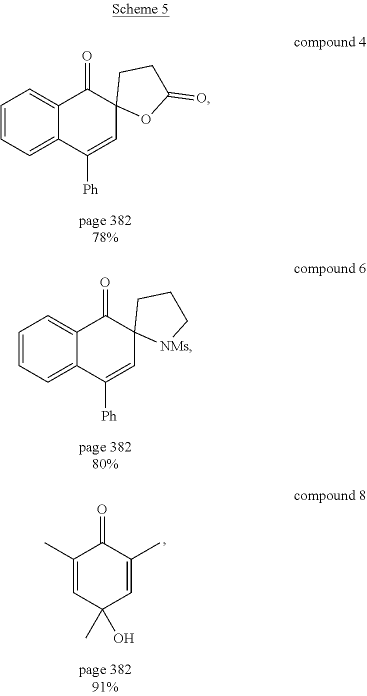

In 2020, the same authors describe in Nature Chemistry 2020, 12, 353-362, the use of the oxidant, sodium hypochlorite, to generate ortho-quinone-methide derivatives from phenolic derivatives, which are then transformed into spiro-epoxide derivatives, for example, see Scheme 6 and 7.

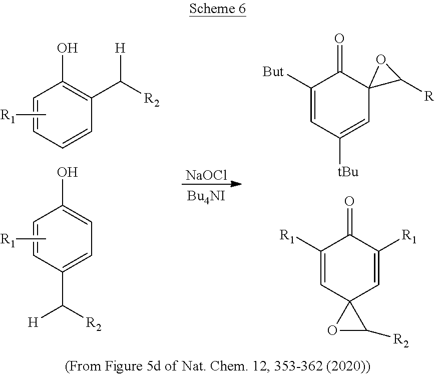

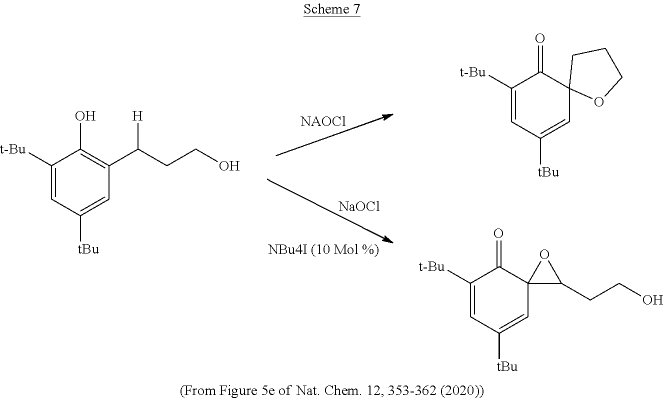 [SMILES: CC(C)(C)c1cc(CCCO)c(O)c(C(C)(C)C)c1 ; "NAOCl" ; "NaOCl" ; CC(C)(C)C1=CC2(OC2CCO)C(=O)C(C(C)(C)C)=C1 ; CC(C)(C)C1=CC2(CCCO2)C(=O)C(C(C)(C)C)=C1]

In these examples, no diiodine derivatives are obtained, and no example uses a 4-hydroxybenzylalcohol derivative as starting material.

Furthermore, by applying various conditions described in this article by Ishihara in 2020, the desired compound 1 is obtained in yields of less than 21% (comparative examples 42 and 43).

The surprising fact of the process of the invention is therefore that the oxidation of compound 2 can be carried out in a single step with much higher yields than are known, and this for a di-iodinated compound.

According to the invention, in the embodiments giving the best results:
Ether-type solvents are used in a two-phase medium, in the presence of a certain percentage of aqueous solution or final water, between 2 and 21%, in particular between 5 and 12%, and even better around 6% (Examples 1, 7 and 8) and a concentration of hypochlorite in the water or aqueous solution of between 0.5M and 1.5M.
The polar organic solvent used is an ether selected from methyl tert-butyl ether (MTBE), tert-amyl methyl ether (TAME), diethyl ether or methyl cyclopentyl ether (Examples 1, 2, 3 and 4).
As an aqueous solution, a buffer solution can be used, for example at a pH between 5 and 8, preferably between 6 and 7, e.g. an acetate buffer solution (Example 11).

Surprisingly, other solvents such as dimethyl carbonate, 2-methyl tetrahydrofuran, isopropyl acetate, n-butanol, ethanol, anisole, toluene, n-heptane, cyclohexane, dimethyl sulfoxide, acetonitrile, N,N-dimethylformamide respectively give substantially lower or zero yields of the desired compound. (Examples 22, 23, 24, 25, 26, 27, 28, 29, 30, 31, 32 and 33 respectively).
Between 2-3, in particular 2-2.5, equivalents of oxidant are used (Example 1).

Surprisingly, other oxidizing agents, such as potassium hydrogen sulfate (Oxone), sodium bismuthate, iodobenzene diacetate (PIDA), tetra-n-butylammonium triiodide (TBAI₃) and bromine with sodium hydroxide do not give the desired compound 1 (Examples 37, 38, 39, 40, 41, 42 and 43 respectively).
A temperature between 0 and 21° C. (Examples 1, 5, 6, 34 and 35).

### Experimental Part

In the description and claims all % are given by weight, temperature is expressed in ° C. or is by default ambient temperature, pressure is atmospheric pressure, unless otherwise indicated. By ambient temperature is meant a temperature usually between about 20° C. and 25° C.

### EXAMPLES ACCORDING TO THE INVENTION

### Example 1 According to the Invention: Best Procedure

The starting 4-hydroxy-3,5-diiodobenzyl alcohol can be prepared from 4-hydroxy benzyl alcohol, according to one of examples 1 to 8 of INNOVERDA patent FR-3,113,904B2.

100 mg (266 μmol) of 4-hydroxy-3,5-diiodobenzyl alcohol is dissolved in 11 mL of methyl tert-butyl ether (MTBE). Separately, 87.5-131 mg (532-798 μmol) sodium hypochlorite pentahydrate is dissolved in 0.7 mL deionized water. Alternatively, 286 μL (532 μmol) of a commercial sodium hypochlorite solution (11-15% active chlorine) is dissolved in 414 μL deionized water. The aqueous fraction is added dropwise over 10 minutes to the organic fraction under vigorous stirring at 21° C. The reaction is stirred for 5 minutes. The reaction is stopped by adding 10 mL of 10% sodium bisulfite solution. This yields 63% 5,7-diiodo-1-oxaspiro[2,5]octa-4,7-dien-6-one (compound 1).

The reaction solution can be used directly, for example for the synthesis of Levothyrox, as is known to the Man skilled in the Art, in particular as described in Tetrahedron Letters, Vol. 38, No. 40, pp. 6965-6968, 1997.

### Additional Examples According to the Invention

### Change of Ether Solvent

Example 2: Proceed as described in Example 1 but use tert-amyl methyl ether instead of methyl tert-butyl ether (MTBE) to obtain 61% of compound 1.

Example 3: Proceed as described in Example 1 but use diethyl ether instead of methyl tert-butyl ether (MTBE) to obtain 61% of compound 1.

Example 4: Proceed as described in Example 1 but using methyl cyclopentyl ether instead of methyl tert-butyl ether (MTBE), 50% of compound 1 is obtained.

### Modification of Reaction Temperature

Proceed as described in Example 1 except for the modification described in Table I below.

TABLE I
Reaction Temperature variation

| | | Yield |
|---|---|---|
| Examples | Modifications | (compound 1) |
| Example 5 | The aqueous fraction containing sodium | 61% |
| | hypochlorite is added at 0° C. | |
| Example 6 | The aqueous fraction containing sodium | 60% |
| | hypochlorite is added at 10° C. | |

### Modification of Concentration of Compound 2:

Proceed as described in Example 1 with the exception of the change described in Table II below.

TABLE II
Change in concentration of compound 2

| | | Yield |
|---|---|---|
| Examples | Modifications | (compound 1) |
| Example 7 | Using 207 mg (550 μmol) of 4-hydroxy- | 65% |
| | 3,5-diiodobenzyl alcohol in 11 mL of methyl | |
| | tert-butyl ether and 181 mg (1.10 mmol) | |
| | of sodium hypochlorite pentahydrate in | |
| | 1.45 mL of demineralized water. | |
| Example 8 | Using 414 mg (1.10 mmol) of 4-hydroxy- | 63% |
| | 3,5-diiodobenzyl alcohol in 11 mL of methyl | |
| | tert-butyl ether and 362 mg (2.20 mmol) | |
| | of sodium hypochlorite pentahydrate in | |
| | 2.89 mL of demineralized water. | |

### Modification of Scale:

Proceeding as described in Example 1 except for the modification described in Table III below.

TABLE III
Modification of scale

| | | Yield |
|---|---|---|
| Examples | Modifications | (compound 1) |
| Example 9 | 1.0 g of 4-hydroxy-3,5-diiodobenzyl | 53% |
| | alcohol is used as starting material. All | |
| | other reagents are increased in proportion | |
| | (×10). | |
| Example 10 | 2.5 g of 4-hydroxy-3,5-diiodobenzyl | 53% |
| | alcohol are used as starting material. All | |
| | other reagents are increased in proportion | |
| | (×25). | |

### Example 11 According to the Invention and Comparative Examples

Changing the Nature of the Aqueous Phase with a Buffer:

Proceed as described in Example 1 with the exception of the modification described in Table IV below.

TABLE IV
Changing the nature of the aqueous phase
with a buffer and pH variation

| | | Yield |
|---|---|---|
| | | (compound |
| Examples | Modifications | 1) |
| Example of | Use of acetate buffer (AcOH/AcONa) 0.1M | 59% |
| invention 11 | pH = 6 instead of demineralized water. | |
| Example of | Use of phosphate buffer (NaH₂PO4/ | 42% |
| invention 12 | Na₂HPO₄) 0.1M pH = 7 instead of | |
| | demineralized water. | |
| Comparative | Use of borate buffer (B(OH)₃/NaOH) | 38% |
| example 13 | 0.1M pH 8.6 instead of demineralized water. | |
| Comparative | Use of acetate buffer (AcOH/AcONa) 0.1M | 29% |
| example 14 | pH = 5 instead of demineralized water. | |
| Comparative | Use of acetate buffer (AcOH/AcONa) 0.1M | 0% |
| example 15 | pH = 4 instead of demineralized water. | |

### Changing the Percentage of Water:

### Example of the Invention 16

100 mg (266 μmol) of 4-hydroxy-3,5-diiodobenzyl alcohol is dissolved in 11 mL of ethyl acetate. Separately, 58.2 mg (354 μmol) sodium hypochlorite pentahydrate is dissolved in 0.7 mL deionized water (final organic/water ratio 94/6). The aqueous fraction is added to the organic fraction with vigorous stirring at 21° C. The reaction is stirred for 5 minutes. The reaction is stopped by adding 10 mL of a 10% sodium bisulfite solution. 41% of 5,7-diiodo-1-oxaspiro[2,5]octa-4,7-dien-6-one (compound 1) is obtained.

Proceed as described in example 16 of the invention above, with the exception of the modification described in Table V below for comparative examples 17 to 21.

TABLE V
Variations in water percentage

| | | Yield |
|---|---|---|
| | | (compound |
| Comparative examples | Modifications | 1) |
| Comparative example 17 | Final organic/water ratio 0/100 | 1% |
| Comparative example 18 | Final organic/water ratio 50/50 | 10% |
| Comparative example 19 | Final organic/water ratio 90/10 | 25% |
| Comparative example 20 | Final organic/water ratio | 9% |
| | 98.5/1.5 | |
| Comparative example 21 | Final organic/water ratio 100/0 | 0% |

### Change of Organic Solvent:

Proceed as described in Comparative Example 16 with the exception of the modification described in Table VI below for Comparative Examples 22 to 33.

TABLE VI
Change of polar organic solvent

| Invention and | | | Yield |
|---|---|---|---|
| comparative | | | (compound |
| examples | Modifications | | 1) |
| Comparative | Carbonate | Use of dimethyl carbonate | 37% |
| example 22 | series | instead of methyl tert- | |
| | | butyl ether. | |
| Comparative | Ether | Use of 2-methyl | 23% |
| example 23 | series | tetrahydrofuran instead of | |
| | | methyl tert-butyl ether. | |
| Comparative | Ester | Use of isopropyl acetate | 31% |
| example 24 | series | instead of methyl tert- | |
| | | butyl ether. | |
| Comparative | Protic | Use of n-butanol instead of | 1% |
| example 25 | series | methyl tert-butyl ether. | |
| Comparative | Protic | Use of ethanol instead of | 0% |
| example 26 | series | methyl tert-butyl ether. | |
| Comparative | Aromatic | Use of anisole instead of | 5% |
| example 27 | series | methyl tert-butyl ether. | |
| Comparative | | Use of toluene instead of | 0% |
| example 28 | | methyl tert-butyl ether. | |
| Comparative | Hydrocarbon | Use of n-heptane instead of | 0% |
| example 29 | series | methyl tert-butyl ether. | |
| Comparative | | Use of cyclohexane instead | 0% |
| example 30 | | of methyl tert- butyl ether. | |
| Comparative | Aprotic | Use of dimethylsulfoxide | 0% |
| example 31 | dipolar | instead of methyl tert- | |
| | series | butyl ether. | |
| Comparative | | Use of acetonitrile instead | 1% |
| example 32 | | of methyl tert-butyl ether. | |
| Comparative | | Use of N,N- | 0% |
| example 33 | | dimethylformamide instead | |
| | | of methyltert-butyl ether. | |

### Modification of Reaction Temperature:

Following on from Examples 5 and 6, the procedure is as described in Example 1, with the exception of the modification described in Table VII below, concerning Comparative Examples 34 and 35.

TABLE VII
TEMPERATURE MODIFICATION

| Comparative | | Yield |
|---|---|---|
| examples | Modifications | (compound 1) |
| Comparative | The aqueous fraction containing | 4% |
| example 34 | hypochlorite is added at −10° C. | |
| Comparative | Aqueous fraction containing hypochlorite | 7% |
| example 35 | is added at 30° C. | |

Comparison with Other Oxidizing Agents:

### Example of the Invention 36

100 mg (266 μmol) of 4-hydroxy-3,5-diiodobenzyl alcohol is dissolved in 11 mL of methyl tert-butyl ether. Separately, 38.0 mg (266 μmol) calcium hypochlorite is dissolved in 0.7 mL deionized water. The aqueous fraction is added dropwise over 10 minutes to the organic fraction with vigorous stirring at 21° C. The reaction is stirred for 5 minutes. The reaction is stopped by adding 10 mL of a 10% sodium bisulfite solution. The result is 38% 5,7-diiodo-1-oxaspiro[2,5]octa-4,7-dien-6-one (compound 1).

Comparative example 37: 100 mg (266 μmol) of 4-hydroxy-3,5-diiodobenzyl alcohol is dissolved in 11 mL of ethyl acetate and 0.7 mL of demineralized water. 482 mg (353 μmol) of potassium hydrogen sulfate is added in one portion to the organic fraction with vigorous stirring at 21° C. The reaction is stirred for 5 minutes. The reaction is stopped by adding 10 mL of 10% sodium bisulfite solution. This yields 0% 5,7-diiodo-1-oxaspiro[2,5]octa-4,7-dien-6-one (compound 1).

Comparative example 38: 100 mg (266 μmol) of 4-hydroxy-3,5-diiodobenzyl alcohol is dissolved in 11 mL ethyl acetate and 0.7 mL deionized water. 117 mg (354 μmol) of sodium bismuthate is added in one portion to the organic fraction with vigorous stirring at 21° C. The reaction is stirred for 5 minutes. The reaction is stopped by adding 10 mL of 10% sodium bisulfite solution. 1% 5,7-diiodo-1-oxaspiro[2,5]octa-4,7-dien-6-one (compound 1) is obtained.

Comparative example 39: 37.8 mg (101 μmol) of 4-hydroxy-3,5-diiodobenzyl alcohol is dissolved in 18 mL of dry acetonitrile. Separately, under argon, 40.8 mg (127 μmol) of iodobenzene diacetate is dissolved in 2 mL of ethyl acetate. Under argon, the small organic fraction is added in one portion to the large organic fraction under strong agitation and at 0° C. Under argon, the reaction is stirred for 30 minutes. The reaction is stopped by adding 10 mL of a 10% sodium bisulfite solution. This yields 2% 5,7-diiodo-1-oxaspiro[2,5]octa-4,7-dien-6-one (compound 1).

Comparative example 40: 100 mg (266 μmol) of 4-hydroxy-3,5-diiodobenzyl alcohol and 36.8 mg (266 μmol) of potassium carbonate are dissolved in 10 mL ethyl acetate and 0.7 mL demineralized water. Separately, 136 mg (266 μmol) of tetra-n-butylammonium triiodide is dissolved in 1 mL of ethyl acetate. The small organic fraction is added in one portion to the large organic fraction with vigorous stirring at 21° C. The reaction is stopped by adding 10 mL of demineralized water. The result is 0% 5,7-diiodo-1-oxaspiro[2,5]octa-4,7-dien-6-one (compound 1).

Comparative example 41: 100 mg (266 μmol) of 4-hydroxy-3,5-diiodobenzyl alcohol is dissolved in 11 mL of ethyl acetate. Separately, 28.4 mg (710 μmol) sodium hydroxide and 56.7 mg (355 μmol) bromine are dissolved in 0.7 mL deionized water and premixed for 2 minutes. The aqueous fraction is added in a single portion to the organic fraction with vigorous stirring at 21° C. The reaction is stopped by adding 10 mL of 10% sodium bisulfite solution. This yields 0% 5,7-diiodo-1-oxaspiro[2,5]octa-4,7-dien-6-one (compound 1).

Comparative example 42: 100 mg (266 μmol) of 4-hydroxy-3,5-diiodobenzyl alcohol and 6.84 mg (26.6 μmol) of tetrabutylammonium iodide are dissolved in 11 mL of ethyl acetate. Separately, 58.2 mg (354 μmol) sodium hypochlorite pentahydrate is dissolved in 0.7 mL deionized water (final organic/water ratio 94/6). The aqueous fraction is added to the organic fraction with vigorous stirring at 21° C. The reaction is stirred for 15 minutes. The reaction is stopped by adding 10 mL of a 10% sodium bisulfite solution. 21% of 5,7-diiodo-1-oxaspiro[2,5]octa-4,7-dien-6-one (compound 1) is obtained.

Comparative example 43: 100 mg (266 μmol) of 4-hydroxy-3,5-diiodobenzyl alcohol is dissolved in 11 mL toluene. Separately, 219 mg (1.33 mmol) sodium hypochlorite pentahydrate is dissolved in 11 mL deionized water (final organic/water ratio 50/50). The aqueous fraction is added to the organic fraction with vigorous stirring at 21° C. The reaction is stirred for 120 minutes. The reaction is stopped by adding 10 mL of a 10% sodium bisulfite solution. The result is 2% 5,7-diiodo-1-oxaspiro[2,5]octa-4,7-dien-6-one (compound 1).

### REFERENCES

1. Grzegorz M. Salamonczyk, Vibha B. Oza and Charles J. Sih, Tetrahedron Letters, 1997, 38, 40, 6965-6968.
2. Halogenated Phenols for diagnostics, antioxidant protection and drug delivery, WO2013/010102 A2.
3. Michael F. McLaughlin, Elisabetta Massolo, Thomas A. Cope, and Jeffrey S. Johnson, Org. Lett. 2019, 21, 6504-6507.
4. Muhammet Uyanik, Niiha Sasakura, Mitsuyoshi Kuwahata, Yasukazu Ejima, and Kazuaki Ishihara, Chem. Lett. 2015, 44, 381-383. doi:10.1246/cl.141130.
5. Muhammet Uyanik, Kohei Nishioka, Ryutaro Kondo, and Kazuaki Ishihara. Chemoselective oxidative generation of ortho-quinone methides and tandem transformations. Nat. Chem. 2020, 12, 353-362. doi:10.1038/s41557-020-0433-4.

1. A process for a direct or single-step synthesis of a compound 5,7-diiodo-1-oxaspiro[2,5]octa-4,7-dien-6-one (compound 1) from 4-Hydroxy-3,5-diiodobenzyl alcohol (compound 2), comprises performing an oxidation reaction of 4-Hydroxy-3,5-diiodobenzyl alcohol with an oxidant comprising a hypochlorite.

2. The process according to claim 1, wherein the oxidation reaction takes place in a two-phase reaction medium comprising a polar organic solvent and an aqueous solution.

3. The process according to claim 2, wherein the polar organic solvent is chosen from a methyl tert-butyl ether (MTBE), a tert-amyl methyl ether (TAME), a diethyl ether and methyl cyclopentyl ether, or a mixture thereof.

4. The process according to claim 2, wherein the aqueous solution is selected from water, demineralized water, er an aqueous buffer, an aqueous buffer with a pH between 5 and 8, or an acetate aqueous buffer.

5. The process according to one claim 1, wherein a relative molar ratio between oxidant and 4-hydroxy-3,5-diiodobenzyl alcohol is between 2 and 3.

6. The process according to claim 2, wherein a ratio by volume of the aqueous solution to the total volume of the aqueous solution and the ether-type polar organic solvent is between 2 and 21%.

7. The process according to claim 2, wherein a temperature of the two-phase reaction medium is between 0 and a room temperature or below the room temperature, the room temperature being defined as between 2° and 25° C.

8. The process according to claim 7, wherein the temperature of the two-phase reaction medium is between 0 and 21° C.

9. The process according to claim 1, wherein a duration of the oxidation reaction is between 1 and 15 minutes.

10. The process according to claim 2, wherein the 4-hydroxy-3,5-diiodobenzyl alcohol is dissolved in the polar organic solvent and the oxidant is dissolved in the aqueous solution.

11. The process according to claim 2, wherein a molar concentration of the hypochlorite oxidant in the aqueous solution is from 0.5M to 1.5M.

12. The process according to claim 2, wherein the aqueous solution is added to the polar organic solution, dropwise, over a period of time of 1 to 15 minutes, with stirring.

13. The process according to claim 1, wherein the hypochlorite is selected from a sodium hypochlorite, a calcium hypochlorite, a potassium hypochlorite, or a mixture thereof.

14. The process according to claim 5, wherein the relative molar ratio between oxidant and 4-hydroxy-3,5-diiodobenzyl alcohol is between 2 and 2.5.

15. The process according to claim 6, wherein ratio by volume of the aqueous solution to the total volume of the aqueous solution and the ether-type polar organic solvent is between 5 and 12%.

16. The process according to claim 9, wherein the duration of the oxidation reaction is between 4 and 10 minutes.

17. The process according to claim 12, wherein the aqueous solution is added to the polar organic solution, dropwise, over a period of time of 4 to 10 minutes, with stirring.
